(19) United States
(12) Patent Application Publication
(10) Pub. No.: US 2021/0252233 A1
(43) Pub. Date: Aug. 19, 2021

(54) PORTABLE MEDICAL SPRAY

(71) Applicant: Chung-Sing Lin, TAINAN (TW)

(72) Inventor: Chung-Sing Lin, TAINAN (TW)

(73) Assignee: Chung-Sing Lin, Tainan (TW)

(21) Appl. No.: 16/792,278

(22) Filed: Feb. 16, 2020

(51) Int. Cl. A61M 11/00; B65D 83/38; A61M 15/08; A61J 1/20

(52) U.S. Cl. CPC A61M 11/007; A61J 1/201; A61M 15/08; B65D 83/382

(57) ABSTRACT

A portable medical spray includes a tubular body having a room defined therein and a canister is located in the room. A needle is located in the room and a nozzle is formed to the top end of the body. The nozzle has a passage defined therethrough and the passage communicates with the room. The passage is located corresponding to the needle. A holding portion is formed to outside of the body. The canister has medicine received therein. A breakable portion is formed to the top end of the canister and located corresponding to the needle. The medicine is ejected through the passage of the nozzle when the canister is pushed and the needle penetrates through the breakable portion of the canister.

### BACKGROUND OF THE INVENTION

### 1. Fields of the Invention

The present invention relates to a medical spray, and more particularly, to a portable medical spray.

### 2. Descriptions of Related Art

The conventional medical spray is used to soothe uncomfortable symptoms caused by rhinitis, stuffy nose, throat ache and the like, and the medical spray is inserted into the patient's nose or mouth to spray medicine onto the infected areas. When spraying the medicine, a compressor and a spray are needed to send medicine to the desired areas. These devices may not be conveniently obtained in remote areas. The present invention intends to provide a portable medicine spray to eliminate the shortcomings mentioned above.

### SUMMARY OF THE INVENTION

The present invention relates to a portable medical spray and comprises a body having a room defined therein. A needle is located in the room. A nozzle is formed to the top end of the body and has a passage defined through the nozzle. The passage communicates with the room and is located corresponding to the needle. A holding portion is formed to outside of the body. A canister is located in the room and has medicine received therein. A breakable portion is formed to the top end of the canister and located corresponding to the needle.

Preferably, the canister contains one-time use volume of the medicine.

Preferably, the canister is made by metal.

Preferably, the breakable portion of the canister is made by rubber.

Preferably, a valve is located in the nozzle, and a knob is connected to the nozzle so as to operate the valve.

The primary object of the present invention is to provide a portable medical spray which is compact for easy carry. The canister can be replaced to include different medicine therein.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the portable medical spray of the present invention;

FIG. 2 is a perspective view to show the portable medical spray of the present invention;

FIG. 3 is a cross sectional view of the portable medical spray of the present invention;

FIG. 4 is a cross sectional view to show that the canister is pushed toward the needle, and the needle penetrates through the breakable portion to eject the medicine in the canister from the nozzle of the portable medical spray of the present invention, and

FIG. 5 is a perspective view to show that the portable medical spray of the present invention is used as a nasal spray.

### DETAILED DESCRIPTION OF THE PREFERRED EMBODIMENT

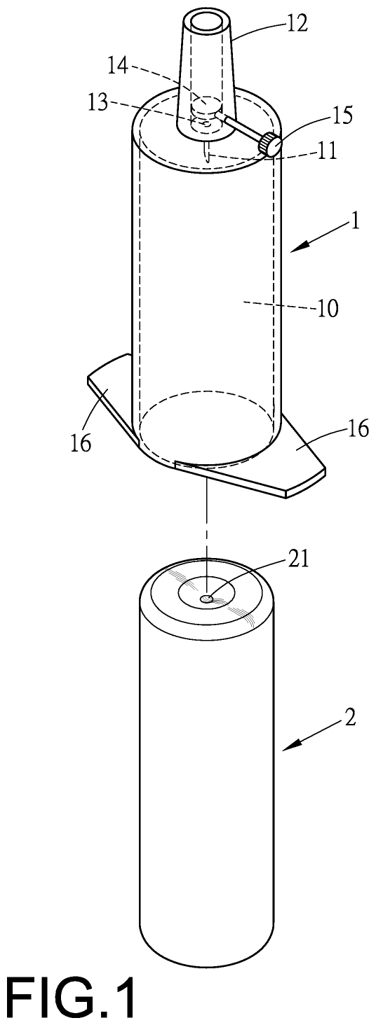

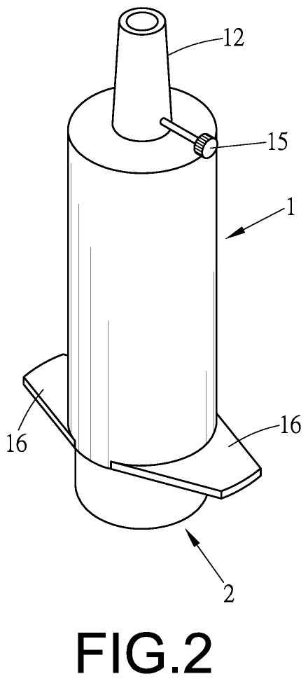

Referring to FIGS. 1 to 3, the portable medical spray of the present invention comprises a tubular body 1 having a room 10 defined therein. A needle 11 is located in the room 10 and a nozzle 12 is formed to the top end of the body 1. A passage 13 is defined through the nozzle 12 and 13 communicates with the room 10 and is located corresponding to the needle 11. A holding portion 16 is formed to outside of the body 1 and includes two finger flanges. A canister 2 is located in the room 10 and has medicine 20 received therein. The medicine 20 is stored in the canister 2 under pressure. A breakable portion 21 is formed to the top end of the canister 2 and located corresponding to the needle 11. Preferably the canister 2 is made by metal, and contains one-time use volume of the medicine 20. The breakable portion 21 of the canister 2 is made by rubber. A valve 14 is located in the nozzle 12 and a knob 15 is connected with a rod which extends through the nozzle 12 and is connected to the valve 14 so that when the user rotates the knob 15, the valve 14 is operated.

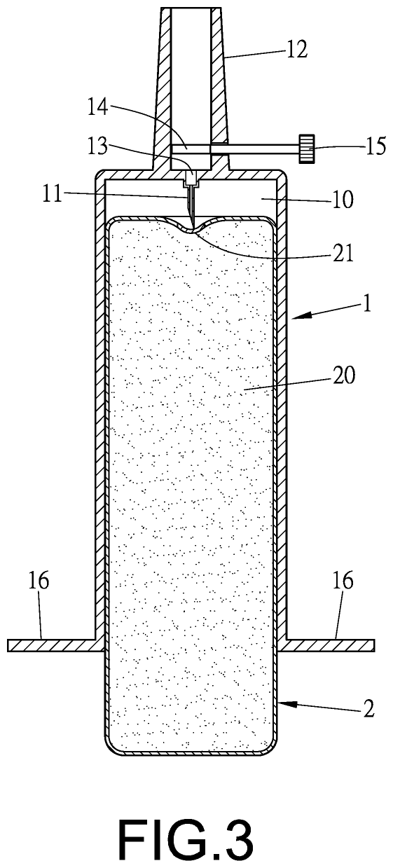

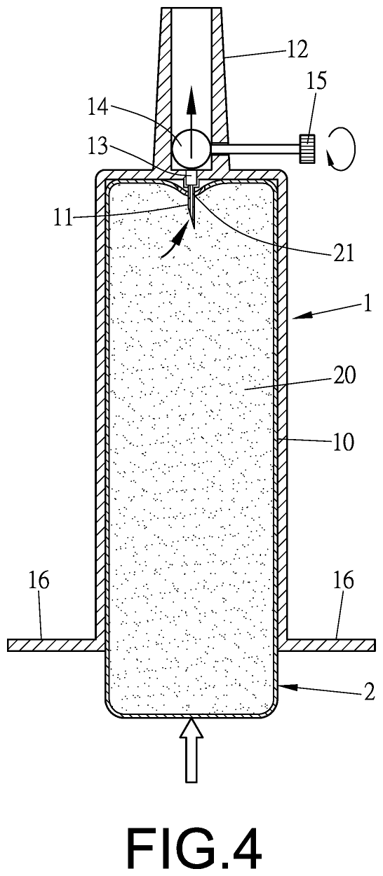

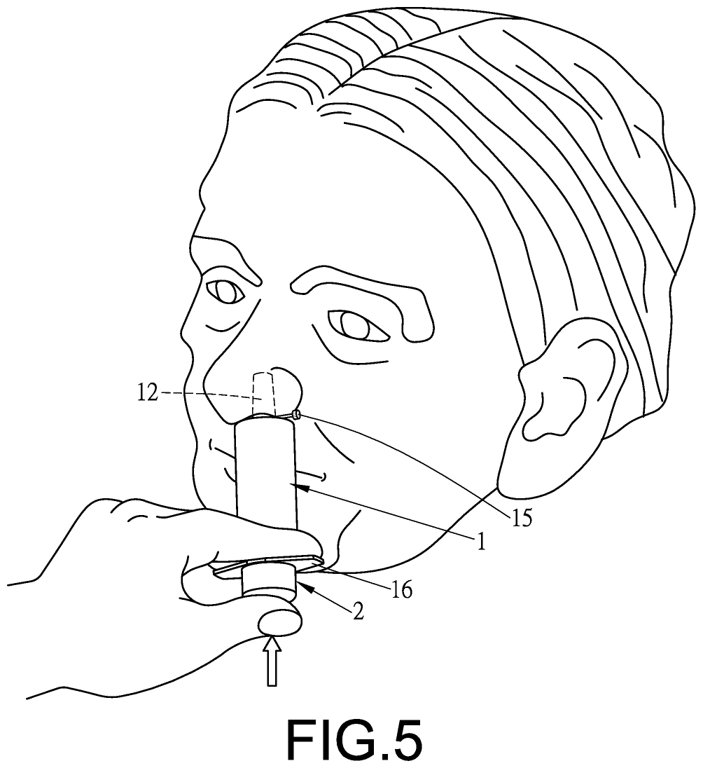

As shown in FIGS. 3 to 5, the canister 2 is inserted into the room 10 of the body 1, and the user may operate the knob 15 to drive the valve 14 to adjust the volume of the medicine 20 in the canister 2 to be ejected. The user holds the body 1 by using two fingers to hold the holding portion 16, and then insert the nozzle 12 into the patient's nose as shown in FIG. 5. Then the canister 2 is pushed toward the needle 11, and the needle 11 penetrates through the breakable portion 21 of the canister 2. The medicine 20 then ejects into the patient's nose through the passage 13 of the nozzle 12. The canister 2 can be replaced with a new one in which different medicine 20 is stored. The portable medical spray of the present invention is compact for easy carry.

While we have shown and described the embodiment in accordance with the present invention, it should be clear to those skilled in the art that further embodiments may be made without departing from the scope of the present invention.

What is claimed is:

1. A portable medical spray comprising:
a body having a room defined therein, a needle located in the room, a nozzle formed to a top end of the body and having a passage defined through the nozzle, the passage communicating with the room and located corresponding to the needle, a holding portion formed to outside of the body, and
a canister located in the room and having medicine received therein, a breakable portion formed to a top end of the canister and located corresponding to the needle.

2. The portable medical spray as claimed in claim 1, wherein the canister contains one-time use volume of the medicine.

3. The portable medical spray as claimed in claim 1, wherein the canister is made by metal.

4. The portable medical spray as claimed in claim 1, wherein the breakable portion of the canister is made by rubber.

5. The portable medical spray as claimed in claim 1, wherein a valve is located in the nozzle, a knob is connected to the nozzle and drives the valve.
